(19) United States
(12) Patent Application Publication
(10) Pub. No.: US 2012/0264920 A1
(43) Pub. Date: Oct. 18, 2012

(54) PROCESSES FOR PURIFICATION OF PROTEINS

(75) Inventors: Chen Wang, Shrewsbury, MA (US); Robert K. Hickman, Worcester, MA (US)

(73) Assignee: ABBOTT LABORATORIES, Abbott Park, IL (US)

(21) Appl. No.: 13/270,443

(22) Filed: Oct. 11, 2011

Related U.S. Application Data
(60) Provisional application No. 61/391,762, filed on Oct. 11, 2010.

(51) Int. Cl. C07K 1/36

(52) U.S. Cl. 530/388.1; 530/417; 530/413; 530/416; 530/387.1; 530/414

(57) ABSTRACT

The invention is directed to a method for purifying a protein. The method involves providing a sample containing the protein, processing the sample through a capture chromatography resin, inactivating viruses in the sample, and processing through at least one depth filter and ion-exchange membrane.

### RELATED APPLICATIONS

This application claims the benefit of priority of U.S. Provisional Application Ser. No. 61/391,762, filed Oct. 11, 2010 which is incorporated herein by reference in its entirety.

### BACKGROUND OF THE INVENTION

The present invention relates generally to methods of purifying proteins.

The economics of large-scale protein purification are important, particularly for therapeutic antibodies, as antibodies make up a large percentage of the therapeutic biologics on the market. In addition to their therapeutic value, monoclonal antibodies, for example, are also important tools in the diagnostic field. Numerous monoclonal antibodies have been developed and used in the diagnosis of many diseases, in diagnosing pregnancy, and in drug testing.

Typical purification processes involve multiple chromatography steps in order to meet purity, yield, and throughput requirements. The steps typically involve capture, intermediate purification or polishing, and final polishing. Affinity chromatography (Protein A or G) or ion exchange chromatography is often used as a capture step. Traditionally, the capture step is then followed by at least two other intermediate purification or polishing chromatography steps to ensure adequate purity and viral clearance. The intermediate purification or polishing step is typically accomplished via affinity chromatography, ion exchange chromatography, or hydrophobic interaction, among other methods. In a traditional process, the final polishing step may be accomplished via ion exchange chromatography, hydrophobic interaction chromatography, or gel filtration chromatography. These steps remove process- and product-related impurities, including host cell proteins (HCP), DNA, leached protein A, aggregates, fragments, viruses, and other small molecule impurities from the product stream and cell culture.

### SUMMARY OF THE INVENTION

Briefly, the present invention is directed, in an embodiment, to a method for purifying a protein comprising providing a sample containing the protein, processing the sample through a capture chromatography resin to provide a first eluate comprising the protein, inactivating viruses in the first eluate to provide an inactivated eluate comprising the protein, processing the inactivated eluate through at least one depth filter to provide a filtered eluate comprising the protein, and processing the filtered eluate through at least one ion-exchange membrane to provide a second eluate comprising the protein.

Further, the invention is directed, in an embodiment, to a method for purifying a protein comprising providing a sample containing the protein, clarifying the sample to provide a clarified sample, processing the clarified sample through a capture chromatography resin to provide a first eluate comprising the protein, inactivating viruses in the first eluate to provide an inactivated eluate comprising the protein, processing the inactivated eluate through at least one depth filter to provide a filtered eluate comprising the protein, processing the filtered eluate through at least one ion-exchange membrane, which is either assembled in series with the depth filter or used in a separate step, to provide a second eluate comprising the protein, processing the second eluate through an additional chromatography resin to provide a third eluate comprising the protein, subjecting the third eluate to nanofiltration to provide a nanofiltered eluate comprising the protein, and subjecting the nanofiltered eluate to ultrafiltration and nanofiltration or diafiltration.

### BRIEF DESCRIPTION OF THE DRAWINGS

FIG. 1 illustrates a schematic of an embodiment of the process.

FIG. 2 illustrates a schematic of another embodiment of the process.

FIG. 3 illustrates a schematic of yet another embodiment of the process.

FIG. 4 illustrates a schematic of yet another embodiment of the process

FIG. 5 illustrates ProSep® Ultra Plus Protein A Capture Chromatography Elution Profiles at 280 nm.

FIG. 6 illustrates ProSep® Ultra Plus Protein A Capture Chromatography Elution Profiles at 302 nm.

FIG. 7 illustrates Phenyl Sepharose® HP Chromatography Profiles at 280 nm.

FIG. 8 illustrates Phenyl Sepharose® HP Chromatography Profiles at 302 nm.

### DETAILED DESCRIPTION OF THE EMBODIMENTS

Reference now will be made in detail to the embodiments of the invention, one or more examples of which are set forth below. Each example is provided by way of explanation of the invention, not a limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment.

Thus, it is intended that the present invention covers such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention.

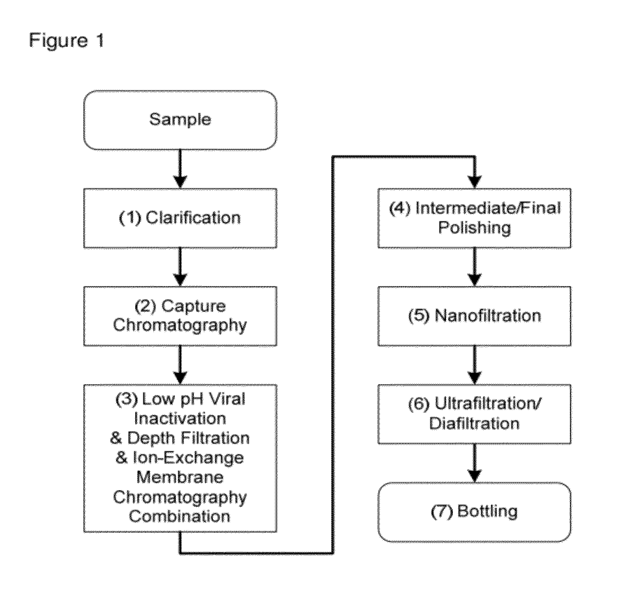

In an embodiment, the present invention comprises a protein purification system and method. Schematic diagrams for embodiments of the present purification system are provided in FIGS. 1-4.

In an embodiment of the invention, a sample which contains a protein is provided. Any sample containing a protein may be utilized in the invention. The sample, which contains a protein, may comprise, for example, cell culture or murine ascites fluid. As an example, the protein may be expressed in Chinese Hamster Ovary (CHO) cells in stirred tank bioreactors. The protein can be any protein, or fragment thereof, known in the art. In various embodiments, the protein is a fusion protein such as an Fc-fusion protein.

In some embodiments, the protein is an antibody. In a particular embodiment, the protein is a monoclonal antibody, or fragment thereof. In some cases, the protein may be a human monoclonal antibody. In other embodiments, the protein is an immunoglobulin G antibody. In an embodiment, the protein may be a veneered immunoglobulin G antibody, a humanized immunoglobulin G antibody, or a recombinant immunoglobulin G antibody. In a particular embodiment, the protein may be an IgG1 immunoglobulin. The protein may, in certain embodiments, be specific for an epitope of human epidermal growth factor receptor (EGFR). The protein may, in another embodiment, be a recombinant, humanized neutralizing monoclonal antibody directed against a unique epitope on IL-13.

In an embodiment of the invention, the sample containing the protein may first be clarified using any method known in the art (see FIGS. 1-4, step 1). The clarification step seeks to remove cells, cell debris, and some host cell impurities from the sample. In an embodiment, the sample may be clarified via one or more centrifugation steps. Centrifugation of the sample may be performed as is known in the art. For example, centrifugation of the sample may be performed using a normalized loading of about 1×10⁻⁸ m/s and a gravitational force of about 5,000×g to about 15,000×g.

In another embodiment, the sample may be clarified via one or more depth filtration steps. Depth filtration refers to a method of removing particles from solution using a series of filters, arranged in sequence, which have decreasing pore size. A depth filter three-dimensional matrix creates a maze-like path through which the sample passes. The principle retention mechanisms of depth filters rely on random adsorption and mechanical entrapment throughout the depth of the matrix. In various embodiments, the filter membranes or sheets may be wound cotton, polypropylene, rayon cellulose, fiberglass, sintered metal, porcelain, diatomaceous earth, or other known components. In certain embodiments, compositions that comprise the depth filter membranes may be chemically treated to confer an electropositive charge, i.e., a cationic charge, to enable the filter to capture negatively charged particles, such as DNA, host cell proteins, or aggregates.

Any depth filtration system available to one of skill in the art may be used in this embodiment. In a particular embodiment, the depth filtration step may be accomplished with a Millistak+® Pod depth filter system, X0HC media, available from Millipore Corporation. In another embodiment, the depth filtration step may be accomplished with a Zeta Plus™ Depth Filter, available from 3M Purification Inc.

In some embodiments, the depth filter(s) media has a nominal pore size from about 0.1 μm to about 8 μm. In other embodiments, the depth filter(s) media may have pore sizes from about 2 μm to about 5 μm. In a particular embodiment, the depth filter(s) media may have pore sizes from about 0.01 μm to about 1 μm. In still other embodiments, the depth filter(s) media may have pore sizes that are greater than about 1 μm. In further embodiments the depth filter(s) media may have pore sizes that are less than about 1 μm.

In some embodiments, the clarification step may involve the use of two or more depth filters arranged in series. The depth filters may be the same or different from one another. In this embodiment, for example, Millistak+® mini D0HC and X0HC filters could be connected in series and used in the clarification step of the invention.

In another embodiment, the clarification step may involve the use of three or more depth filters. In one embodiment, the clarification step may involve the use of multiple (e.g., ten) units of depth filters arranged in parallel. In this embodiment, the multiple units of depth filters may be Millipore® X0HC filters.

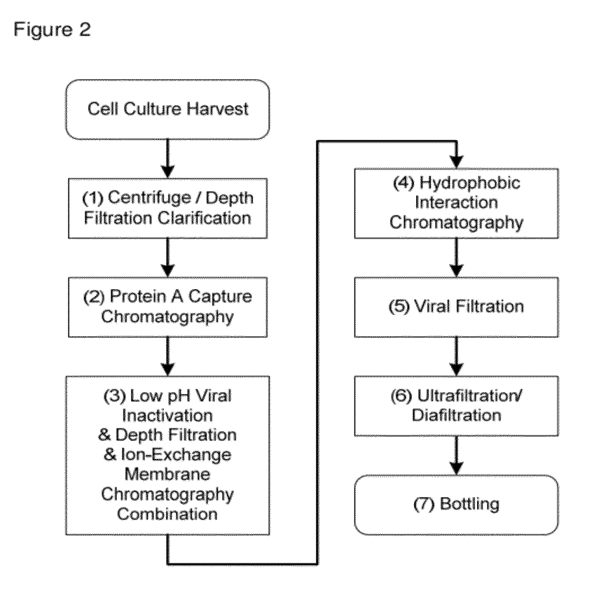

In a particular embodiment, the clarification step may be accomplished through the use of centrifugation followed by X0HC depth filtration, performed in series (FIGS. 2-4, step 1).

In another embodiment, the sample may be clarified via a microfiltration or ultrafiltration membrane in tangential flow filtration (TFF) mode. Any TFF clarification processes known in the art may be utilized in this embodiment. TFF designates a membrane separation process in cross-flow configuration, driven by a pressure gradient, in which the membrane fractionates components of a liquid mixture as a function of particle and/or solute size and structure. In clarification, the selected membrane pore size allows some components to pass through the pores with the water while retaining the cells and cell debris above the membrane surface. In an embodiment, the TFF clarification may be conducted using, for example, a 0.1 μm or 750 kD molecular weight cutoff, 5-40 psig, and temperatures of from about 4° C. to about 60° C. with polysulfone membranes.

In an embodiment of the invention, the clarification step may involve treatment of the sample with a detergent. The detergent utilized may be any detergent known to be useful in protein purification processes. In an embodiment, the detergent may be applied to the sample at a low level and the sample then incubated for a sufficient period of time to inactivate enveloped mammalian viruses. The level of detergent to be applied, in an embodiment, may be from about 0 to about 1% (v/v). In another embodiment, the level of detergent to be applied may be from about 0.05% to about 0.7% (v/v). In yet another embodiment, the level of detergent to be applied may be about 0.5% (v/v). In a particular embodiment, the detergent may be polysorbate 80 (Tween® 80), available from Sigma-Aldrich, Inc., or Triton® X-100, available from Roche Diagnostics GmbH.

Any combination of these or other clarification processes which are known in the art can be utilized as the clarification step of the invention.

In an embodiment, following the clarification step of the invention, the sample may be subjected to a chromatography capture step (see FIGS. 1-4, step 2). The capture step is designed to separate the target protein from other impurities present in the clarified sample. Often, the capture step reduces host cell protein (HCP), host cell DNA, and endogenous virus or virus-like particles in the sample. The chromatography technique utilized in this embodiment may be any technique known in the art to be used as a capture step. In an embodiment, the sample may be subjected to affinity chromatography, ion exchange chromatography, mixed-mode chromatography, or hydrophobic interaction chromatography as a capture step.

In a particular embodiment of the invention, affinity chromatography may be utilized as the capture step. Affinity chromatography makes use of specific binding interactions between molecules. A particular ligand is chemically immobilized or “coupled” to a solid support. When the sample is passed over the resin, the protein in the sample, which has a specific binding affinity to the ligand, becomes bound. After other sample components are washed away, the bound protein is then stripped from the immobilized ligand and eluted, resulting in its purification from the original sample.

In this embodiment of the invention, the affinity chromatography capture step may comprise interactions between an antigen and an antibody, an enzyme and a substrate, or a receptor and a ligand. In a particular embodiment of the invention, the affinity chromatography capture step may comprise protein A chromatography, protein G chromatography, protein A/G chromatography, or protein L chromatography.

In a certain embodiment, protein A affinity chromatography may be utilized in the capture step of the invention (see FIGS. 2-4, step 2). Protein A affinity chromatography involves the use of a protein A, a bacterial protein that demonstrates specific binding to the non-antigen binding portion of many classes of immunoglobulins. The protein A resin utilized may be any protein A resin. In an embodiment, the protein A resin may be selected from the MabSelect™ family of resins, available from GE Healthcare Life Sciences. In another embodiment, the protein A resin may be a ProSep® Ultra Plus resin, available from Millipore Corporation. Any column available in the art may be utilized in this step. In a particular embodiment, the column may be a column packed with MabSelect™ resin, available from GE Healthcare Life Sciences, or a column (e.g. Quickscale column) packed with ProSep® Ultra Plus resin, available from Millipore Corporation.

If protein A affinity is utilized as the chromatography step, the column may have an internal diameter of about 35 cm with a column length of 20 cm. In other embodiments, the column length may be from about 5 cm to about 35 cm. In still another embodiment, the column length may be from about 10 cm to about 20 cm. In yet another embodiment, the column length may be 5 cm or larger. In an embodiment, the internal diameter of the column may be from about 0.5 cm to about 100 or 200 cm. In another embodiment, the internal diameter of the column may be from about 10 cm to about 50 cm. In still another embodiment, the internal diameter of the column may be 15 cm or larger.

The specific methods used for the chromatography capture step, including flow of the sample through the column, wash, and elution, depend on the specific column and resin used and are typically provided by the manufacturers or are known in the art. As used herein, the term “processed” may describe the process of flowing or passing a sample through a chromatography column, resin, membrane, filter, or other mechanism, and shall include a continuous flow through each mechanism as well as a flow that is paused or stopped between each mechanism.

Following the chromatography capture step, the eluate may be subjected to a combination processing step. This combination step may, in an embodiment, comprise viral inactivation followed by processing through one or more depth filers and ion-exchange membranes (see FIGS. 1-4, step 3). In an embodiment, the depth filtration and ion-exchange membrane may be designed as a filter train, in series.

In an embodiment, the viral inactivation step may comprise low-pH viral inactivation. In one aspect, use of a high concentration glycine buffer at low pH for elution may be employed, without further pH adjustment, in a final eluate pool in the targeted range for low-pH viral inactivation. Alternatively, acetate or citrate buffers may be used for elution and the eluate pool may then be titrated to the proper pH range for low-pH viral inactivation. In an embodiment, the pH is from about 2.5 to about 4. In a further embodiment, the pH is from about 3 to about 4.

In an embodiment, once the pH of the eluate pool is lowered, the pool is incubated for a length of time from about 15 to about 90 minutes. In a particular embodiment, the low-pH viral inactivation step may be accomplished via titration with 0.5 M phosphoric acid to obtain a pH of about 3.5 and the sample may then be incubated for a time period between about 60 minutes and 90 minutes.

After the low-pH viral inactivation step, the inactivated eluate pool may be neutralized to a higher pH. In an embodiment, the neutralized, higher pH may be a pH of from about 5 to about 10. In another embodiment, the neutralized, higher pH may be a pH of from about 8 to about 10. In yet another embodiment, the neutralized, higher pH may be a pH of from about 6 to about 10. In yet another embodiment, the neutralized, higher pH may be a pH of from about 6 to about 8. In yet another embodiment, the neutralized, higher pH may be a pH of about 8.0.

In an embodiment, the pH neutralization may be accomplished using 3.0 M trolamine or another buffer known in the art. The conductivity of the inactivated eluate pool may then be adjusted with purified or deionized water. In an embodiment, the conductivity of the inactivated eluate pool may be adjusted to from about 0.5 to about 50 mS/cm. In another embodiment, the conductivity of the inactivated eluate pool may be adjusted to from about 4 to about 6 mS/cm. In a particular embodiment, the conductivity of the inactivated eluate pool may be adjusted to from about 5.0 mS/cm.

In alternative embodiments, the viral inactivation aspect of the combination processing step may be carried out using other methods known in the art. For example, the viral inactivation step may comprise, in various embodiments, treatment with acid, detergent, solvent, chemicals, nucleic acid cross-linking agents, ultraviolet light, gamma radiation, heat, or any other process known in the art to be useful for this purpose.

Following viral inactivation and neutralization, the inactivated eluate pool may be processed through one or more depth filters, as fully described above, and one or more ion-exchange membranes, hydrophobic membranes, or mixed-mode membranes, provided as a filter train or in series.

The depth filtration aspect of the combination step may comprise one or more types of depth filters. In an embodiment, the depth filtration aspect of the combination step may comprise more than one unit of depth filters. These depth filters may, in an embodiment, be Millipore® X0HC filters. One of skill in the art will recognize that selection of the type and number of filters used will depend on the volume of sample being processed.

The ion-exchange aspect of the combination step can be any ion-exchange process known in the art. In an embodiment, this step comprises a membrane chromatography capsule. In an embodiment, Chromasorb™ Membrane Adsorber may be utilized.

In a particular embodiment, the chromatography aspect of the step comprises a Q membrane chromatography capsule. In an embodiment, the Q membrane chromatography capsule may comprise Mustang® Q membrane chromatography capsule (available from Pall Corporation) or Sartobind® Q (available from Sartorius Stedim Biotech GmbH). In an embodiment, the Q membrane chromatography capsule is operated in flow-through mode.

Each of the depth filter and ion-exchange membrane steps may, in an embodiment, be followed by a capsule filtration step. For example, the capsule filtration step may comprise a Sartopore® 2 capsule filter, available from Sartorius Stedim Biotech GmbH.

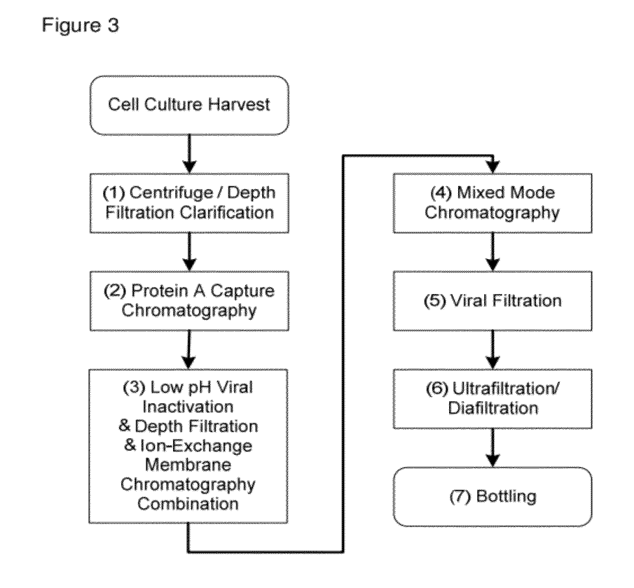

Following the combination processing step, the sample may be subjected to an intermediate/final polishing step (FIGS. 1-4, step 4). This step may, in an embodiment, comprise an additional chromatography step. Any form of chromatography known in the art may be acceptable. For example, in an embodiment, the intermediate/final polishing step may comprise a mixed-mode (also known as multimodal) chromatography step (FIG. 3, step 4). The mixed-mode chromatography step utilized in this invention may utilize any mixed-mode chromatography process known in the art. Mixed mode chromatography involves the use of solid phase chromatographic supports in resin, monolith, or membrane format that employ multiple chemical mechanisms to adsorb proteins or other solutes. Examples useful in the invention include, but are not limited to, chromatographic supports that exploit combinations of two or more of the following mechanisms: anion exchange, cation exchange, hydrophobic interaction, hydrophilic interaction, thiophilic interaction, hydrogen bonding, pi-pi bonding, and metal affinity. In particular embodiments, the mixed-mode chromatography process combines: (1) anion exchange and hydrophobic interaction technologies; (2) cation exchange and hydrophobic interaction technologies; and/or (3) electrostatic and hydrophobic interaction technologies.

In an embodiment, the mixed-mode chromatography step may be accomplished by using a column and resin such as the Capto® adhere column and resin, available from GE Healthcare Life Sciences. The Capto® adhere column is a multimodal medium for intermediate purification and polishing of monoclonal antibodies after capture. In a particular embodiment, the mixed-mode chromatography step may be conducted in flow-through mode. In other embodiments, the mixed-mode chromatography step may be conducted in bind-elute mode.

In other embodiments, the mixed-mode chromatography step may be accomplished by using one or more of the following systems: Capto® MMC (available from GE Healthcare Life Sciences), HEA HyperCel™ (available from Pall Corporation), PPA HyperCel™ (available from Pall Corporation), MBI HyperCel™ (available from Pall Corporation), MEP HyperCel™ (available from Pall Corporation), Blue Trisacryl M (available from Pall Corporation), CFT™ Ceramic Fluoroapatite (available from Bio-Rad Laboratories, Inc.), CHT™ Ceramic Hydroxyapatite (available from Bio-Rad Laboratories, Inc.), and/or ABx (available from J. T. Baker). The specific methods used for the mixed-mode chromatography step may depend on the specific column and resin utilized, and are typically supplied by the manufacturer or are known in the art.

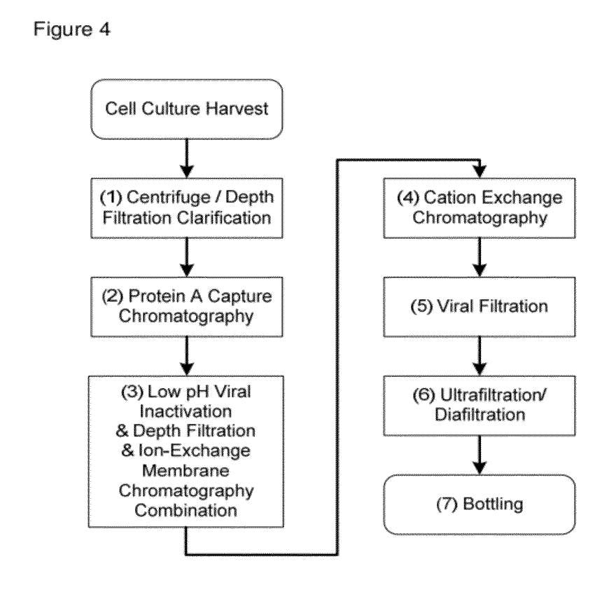

In another embodiment, the intermediate/final polishing step may comprise a cation exchange chromatography (FIG. 4, step 4). The cation exchange chromatography step utilized in this invention may use any cation exchange chromatography process known in the art. In an embodiment, the cation exchange chromatography step may be accomplished by using a column packed with Poros XS resin (Life Technologies). In a particular embodiment, the cation exchange chromatography step may be operated in bind-elute mode.

Each column utilized in the process may be large enough to provide maximum throughput capacity and economies of scale. For example, in certain embodiments, each column can define an interior volume of from about 1 L to about 1500 L, of from about 1 L to about 1000 L, of from about 1 L to about 500 L, or of from about 1 L to about 250 L. In some embodiments, the mixed-mode or cation exchange column may have an internal diameter of about 1 cm and a column length of about 7 cm. In other embodiments, the internal diameter of the mixed-mode or cation exchange column may be from about 0.1 cm to about 100 cm, from 0.1 to 50 cm, from 0.1 cm to about 10 cm, from about 0.5 cm to about 5 cm, from about 0.5 cm to about 1.5 cm, or may be about 1 cm. In an embodiment, the column length of the mixed-mode or cation exchange column may be from about 1 to about 50 cm, from about 1 to about 20 cm, from about 5 to about 10 cm, or may be about 7 cm.

In some embodiments, the inventive systems are capable of handling high titer concentrations, for example, concentrations of about 5 g/L, about 6 g/L, about 7 g/L, about 8 g/L, about 9 g/L, about 10 g/L, about 12.5 g/L, about 15 g/L, about 20 g/L, about 25 g/L, concentrations of from about 1 g/L to about 5 g/L, concentrations of from about 5 g/L to about 10 g/L, concentrations of from about 5 g/L to about 12.5 g/L, concentrations of from about 5 g/L to about 15 g/L, concentrations of from about 5 g/L to about 20 g/L, concentrations of from about 5 g/L to about 55 g/L, or concentrations of from about 5 g/L to about 100 g/L. For example, some of the systems are capable of handling high antibody concentrations and, at the same time, processing from about 200 L to about 2000 L culture per hour, from about 400 L culture to about 2000 L per hour, from about 600 L to about 1500 L culture per hour, from about 800 L to about 1200 L culture per hour, or greater than about 1500 L culture per hour.

In an embodiment, the intermediate/final polishing step may be accomplished via one or more membrane adsorbers or monoliths. Membrane adsorbers are thin, synthetic, microporous or macroporous membranes that are derivatized with functional groups akin to those on the equivalent resins. On their surfaces, membrane adsorbers carry functional groups, ligands, interwoven fibers, or reactants capable of interacting with at least one substance in contact within a fluid phase moving through the membrane by gravity. The membranes are typically stacked 5 to 15 layers deep in a comparatively small cartridge to generate a much smaller footprint than columns with similar outputs. The membrane adsorber utilized herein may be a membrane ion-exchanger, mixed-mode ligand membrane and/or hydrophobic membrane.

In an embodiment, the membrane adsorber utilized may be ChromaSorb™ Membrane Adsorber, available from Millipore Corporation. ChromaSorb™ Membrane Adsorber is a membrane-based anion exchanger designed for the removal of trace impurities including HCP, DNA, endotoxins, and viruses for MAb and protein purification. Other membrane adsorbers that could be utilized include Sartobind® Q (available from Sartorium BBI Systems GmbH), Sartobind® S (available from Sartorium BBI Systems GmbH), Sartobind® C (available from Sartorium BBI Systems GmbH), Sartobind® D (available from Sartorium BBI Systems GmbH), Sartobind® Phenyl (available from Sartorium BBI Systems GmbH), Sartobind® IDA (available from Sartorium BBI Systems GmbH), Pall Mustang® (available from Pall Corporation), or any other membrane adsorber known in the art.

As set forth above, monoliths may be utilized in the intermediate/final polishing step of the invention. Monoliths are one-piece porous structures of uninterrupted and interconnected channels of specific controlled size. Samples are transported through monoliths via convection, leading to fast mass transfer between the mobile and stationary phase. Consequently, chromatographic characteristics are non-flow dependent. Monoliths also exhibit low backpressure, even at high flow rates, significantly decreasing purification time. In an embodiment, the monolith may be an ion-exchange or mixed-mode ligand-based monolith. In one aspect, the monolith utilized may include CIM® monoliths (available from BIA separations), UNO® monoliths (available from Bio-Rad Laboratories, Inc.) or ProSwift® or IonSwift™ monoliths (available from Dionex Corporation).

In still another embodiment, the intermediate/final polishing step may be accomplished via an additional depth filtration step rather than by using membrane adsorbers, monoliths, or a mixed-mode column. In this embodiment, the depth filtration utilized for intermediate/final polishing may be a CUNO Zeta Plus VR® depth filter. In this embodiment, the depth filter may serve the purpose of intermediate/final polishing as well as viral clearance.

In a particular embodiment, the intermediate/final polishing step may be a hydrophobic interaction chromatography step (FIG. 2, step 4). In an embodiment, this step may use Phenyl Sepharose® High Performance hydrophobic interaction resin and a Chromaflow® Acrylic chromatography column, each available from GE Healthcare. Phenyl Sepharose® HP resins are based on rigid, highly cross-linked, beaded agarose with a mean particle diameter of 34 μm. The functional groups are attached to the matrix via uncharged, chemically stable ether linkages resulting in a hydrophobic medium with minimized ionic properties. In this embodiment, the sample may be filtered through a Sartopore® capsule filter prior to loading onto the column.

If hydrophobic interaction chromatography is used in the intermediate/final polishing step, the internal diameter of the column may be between about 10 and 100 cm. In a particular embodiment, the internal diameter may be about 60 cm. The height of the column, in an embodiment, may be between about 10 and 20 cm. In an embodiment, the height of the column is about 15 cm.

Following the intermediate/final polishing chromatography step, the eluate pool may be subjected to a nanofiltration step (see FIGS. 1-4, step 5). In an embodiment, the nanofiltration step is accomplished via one or more nanofilters or viral filters. The filters may be any known in the art to be useful for this purpose and may include, for example, Millipore Pellicon® or Millipak® filters or Sartorius Vivaspin® or Sartopore® filters. In a particular embodiment, the nanofiltration step may be accomplished via a filter train comprised of a prefilter and a nanofilter or viral filters. As an example, the filter train may be comprised of two Pall 0.15 m² 0.1 μm Fluorodyne® II PVDF capsule filters available from Pall Corporation, as protecting filters for two 20-inch Sartorius Virosart® CPV filters, available from Sartorius Stedim Biotech GmbH, in parallel. In another example, the filter train may be comprised of one (0.17 m²) 0.1 μm Maxicap® prefilters and two 20-inch Virosart® CPV filters, both from Sartorius Stedim Biotech GmbH. One of skill in the art will understand that the selection of types and numbers of filters will be dependent on the volume of sample being processed.

As shown in FIGS. 1-4, step 6, the nanofiltration step may be optionally followed by ultrafiltration/diafiltration (UF/DF), to achieve the targeted drug substance concentration and buffer condition before bottling. In an embodiment, this can be accomplished by the use of filters. The filters may be any known in the art to be useful for this purpose and may include, for example, Millipore Pellicon®, Millipak®, or Sartopore® filters. In a particular embodiment, the UF/DF may be accomplished via three Millipore® Pellicon® 2 Biomax UF Modules with a 30 kD molecular weight cut off and 2.5 m² surface area each, optionally followed by filtration through a Sartopore® 2, 800 sterile capsule filter. The nanofiltration and UF/DF steps can be combined or replaced by any process(es) known in the art known to provide a purified protein that is acceptable for bottling (FIGS. 1-4, step 7). Prior to bottling, the samples may, in an embodiment, be pumped through a Millipak® 200 0.22 μm filter into pre-sterilized, pyrogen-free polyethylene terephthalate glycol (PETG) containers.

The following examples describe various embodiments of the present invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples.

### Example 1

Generally speaking, a protein sample (MAb A) was purified from a cell culture supernatant through a series of recovery, capture, and purification steps. The primary recovery steps involved centrifugation and depth filtration. The capture steps involved protein A chromatography, followed by viral inactivation, depth filtration, and Mustang® Q membrane chromatography. The fine purification steps involved hydrophobic interaction chromatography, nanofiltration, and ultrafiltration/diafiltration. The final product was then filtered, bottled, and frozen. The recovery and capture operations were performed at ambient temperature. The fine purification steps were performed at a temperature of 17±2° C., unless otherwise specified. Three batches of 3000 L bioreactor harvest of MAb A were purified through this process.

### Primary Recovery

Primary recovery by centrifugation and depth filtration was used to remove cells and cell debris from the production bioreactor tank. An Alfa-Laval BTUX 510 centrifuge was utilized for this process step. A 3000 L production bioreactor served as the feed tank to a continuous flow disc stack centrifuge. The centrifuge was run at about 5200 rμm at a feed rate of 28 L/minute. The centrifuged harvest was subsequently passed through a filter train that consisted of ten 1.1 m² Millipore® X0HC media Pod units. After the contents of the bioreactor were depth filtered, the filter train was subsequently rinsed with 200 kg of 25 mM Tris, 100 mM sodium chloride, pH 7.2, and then air blown to remove the remaining filtrate. Centrifugation and filtration of the harvest were performed as a single unit operation. The filtrate was collected in a 3000 L harvest tank, chilled to 4-12° C., and held for up to 5 days. In one run, the centrifuge was not utilized and a series of Pod filters was instead used to process the material. A total of fifteen D0HC and ten X0HC filters were used to clarify about 3000 L harvest materials. Again, the filter train was subsequently rinsed with 200 kg of 25 mM Tris, 100 mM sodium chloride, pH 7.2, and then air blown to remove the remaining filtrate. The clarification yield when using the depth filter only was similar to that using both centrifuge and depth filter. Overall, the average harvest step yield was 91% with an average harvest concentration of 1.85 g/L. The results of the harvest centrifugation and filtration operations are summarized in Table 1.

TABLE 1
Summary of Primary Recovery Operations

Run #

| | 1 | 2 | 3 | AVG | STD |
|---|---|---|---|---|---|
| Reactor End Conc. (g/L) | 2.07 | 2.07 | 2.02 | 2.05 | 0.03 |
| Reactor End Volume (kg) | 2903 | 2923 | 2937 | 2921 | 17 |
| Harvest End Conc. (g/L) | 1.87 | 1.79 | 1.88 | 1.85 | 0.05 |
| Harvest End Volume (kg) | 2931 | 3013 | 2895 | 2946 | 60 |
| Harvest Step Yield (%) | 91 | 89 | 92 | 91 | 2 |

### Protein A Capture Chromatography

Protein A chromatography was used to capture the protein from the clarified harvest and to reduce the amount of process-related impurities. ProSep® Ultra Plus resin (Millipore) and a Quickscale chromatography column (Millipore) were utilized for this process step. The protein A capture column was 35 cm in diameter with a target height of 20 cm (bed volume 19.2 L). The loading limit for MAb A in the column was 42 grams of sample per liter of protein A resin. Seven cycles were completed for each batch. The step was performed at ambient temperature and used a 3-step linear velocity loading of 720 cm/hr up to 36 g/L, 480 cm/hr up to 39 g/L, and 240 cm/hr up to 42 g/L. The column was equilibrated with 25 mM Tris, 100 mM sodium chloride, pH 7.2, and loaded with clarified harvest.

After loading, the column was washed to baseline absorbance (A₂₈₀) with equilibration buffer. A second wash of 20 mM sodium citrate/citric acid, 0.5 M sodium chloride, pH 6.0, was utilized in order to reduce the amount of process-related impurities. A third wash of equilibration buffer brought the optical density (OD), pH, and conductivity back to baseline. The product was eluted from the column with 0.1 M acetic acid, pH 3.5. The eluate was collected from 1 OD on the front to 1 OD on the tail at 280 nm with a 1 cm path length. For each cell culture batch, the column was cycled six additional times to process approximately the 5500 g of crude protein that was expected. Between each cycle, the column was regenerated with 0.2 M acetic acid. The eluate pool was held up to 5 days, chilled to 4-12° C., before proceeding to the low pH virus inactivation step.

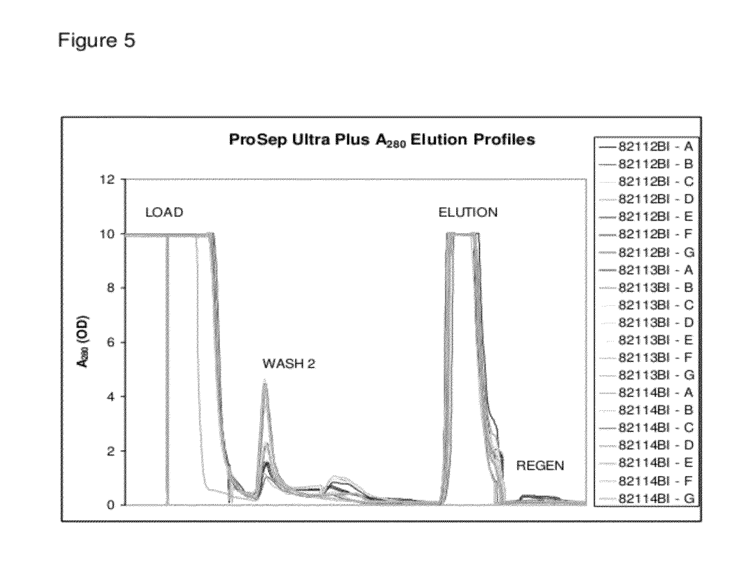

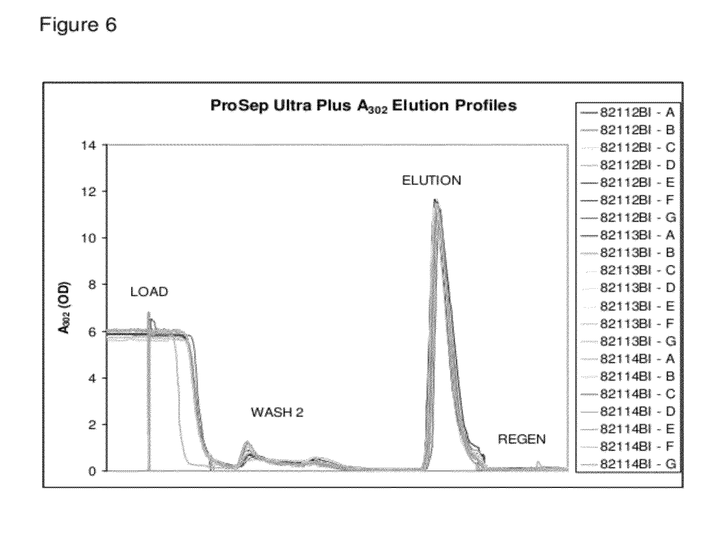

Operational data and yields for the Protein A capture step are shown in Table 2. Average column loadings were approximately 42 g of protein per L of resin per cycle with the exception of the seventh cycle for each batch, which was partially loaded using the remaining load volume. Average yield for the Protein A capture step was 90%. Capture column operations were consistent in regard to elution chromatographic profiles. Overlays are illustrated in FIGS. 5 and 6.

TABLE 2
Summary of ProSep ® Ultra Plus Protein A Capture Chromatography

| | Run # | | | | |
|---|---|---|---|---|---|
| | 1 | 2 | 3 | AVG | STD |
| Total Load Volume (kg) | 2931 | 3013 | 2895 | 2946 | 60 |
| Column Load Conc. (g/L) | 1.87 | 1.79 | 1.88 | 1.85 | 0.05 |
| Eluate Pool Volume (kg) | 245 | 247 | 229 | 240 | 10 |
| Eluate Pool Conc. (g/L) | 20.24 | 19.71 | 21.09 | 20.35 | 0.70 |
| Step Yield (%) | 91 | 91 | 89 | 90 | 1 |

### Viral Inactivation, Depth Filtration, and Q Membrane Chromatography

The Protein A eluate pool was subjected to low pH to inactivate adventitious viruses that may have been present. The step was performed at ambient temperature. The low pH inactivation step was performed by adjusting the pH of the eluate pool to 3.5±0.1 (measured at 25° C.) with 0.5 M phosphoric acid. After a hold period of 60-90 minutes, the inactivated material was neutralized to pH 8.0±0.1 (measured at 25° C.) using 3.0 M trolamine and diluted with purified water to a conductivity of 5.0±0.5 mS/cm. After neutralization, the pH inactivated material was passed through a filter train into a holding tank. The filter train was made of two components. The first consisted of six 1.1 m² Millipore® X0HC media Pod units and the second was a 780 mL Pall Mustang® Q Chromatography Capsule. Average loading over the Mustang® Q capsule was 6.3 g of protein per mL of Q capsule. After depth filtration, and again after Q membrane processing, the sample was flowed through a Sartopore® 2 20-inch (0.45 μm+0.2 μm) capsule filter. After the contents of the feed tank were filtered, the filter train was subsequently rinsed with approximately 100 kg of 25 mM trolamine and 40 mM sodium chloride. The effluent was held at ≦22° C. for up to 1 day. In other cases, the effluent was chilled to ≦8° C. and held up to 3 days before proceeding to the Phenyl Sepharose® HP chromatography step.

The results of the low pH inactivation and filtration operations are summarized in Table 3. Average loading over the Mustang® Q capsule was 6.3 g of protein per mL of Q capsule (equivalent to 409 mL of protein per mL of Q capsule). The three runs had an average step yield of 96%.

TABLE 3
Summary of Viral Inactivation, Depth Filtration, and Q Membrane
Chromatography Operations

| | Run # | | | | |
|---|---|---|---|---|---|
| | 1 | 2 | 3 | AVG | STD |
| Start Volume (kg) | 245 | 247 | 229 | 240 | 10 |
| pH, Initial (Viral | 4.0 | 4.1 | 4.1 | 4.1 | 0.1 |
| Inactivation) | | | | | |
| pH, Final (Viral | 3.5 | 3.6 | 3.6 | 3.6 | 0.1 |
| Inactivation) | | | | | |
| 0.5M Phosphoric Acid | 6.7 | 7.1 | 7.0 | 6.9 | 0.2 |
| Added (kg) | | | | | |
| pH, Initial | 3.6 | 3.6 | 3.6 | 3.6 | 0.0 |
| pH, Final | 7.9 | 7.9 | 7.9 | 7.9 | 0.0 |
| 3.0M Trolamine Added | 16.8 | 16.3 | 14.8 | 16.0 | 1.0 |
| (kg) | | | | | |
| Conductivity, Initial | 6.4 | 6.5 | 6.7 | 6.5 | 0.1 |
| (mS/cm) | | | | | |
| Conductivity, Final | 5.4 | 5.4 | 5.4 | 5.4 | 0.0 |
| (mS/cm) | | | | | |
| USP-PW Added (kg) | 54.7 | 59.7 | 54.0 | 56.1 | 3.1 |
| Mustang ® Q Loading | 6.4 | 6.2 | 6.2 | 6.3 | 0.1 |
| (g of sample/mL of Q | | | | | |
| capsule) | | | | | |
| Filter Train Rinse Volume | 54.8 | 109.4 | 108.2 | 90.8 | 31.2 |
| (kg) | | | | | |
| Final Pool (kg) | 378.0 | 439.5 | 413 | 410 | 31 |
| Final Concentration (g/L) | 11.93 | 10.87 | 10.85 | 11.22 | 0.62 |
| Step Yield (%) | 91 | 100.4 | 97.1 | 96 | 5 |

### Hydrophobic Interaction Chromatography

Phenyl Sepharose® HP chromatography was used to reduce the amount of process-related impurities and aggregated antibody that might be present in the Q membrane effluent. Prior to this polishing step, the Q membrane effluent was diluted with 2.2 M ammonium sulfate and 40 mM sodium phosphate, pH 7.0, to contain a target concentration of 1.0 M ammonium sulfate and 18 mM sodium phosphate and then filtered through a Sartopore® 2 10-inch (0.45 μm+0.2 μm) capsule filter prior to loading onto the column.

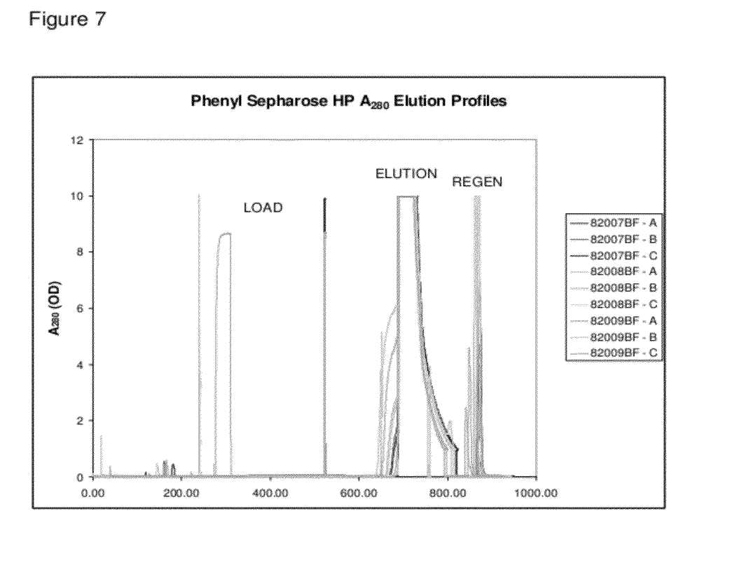

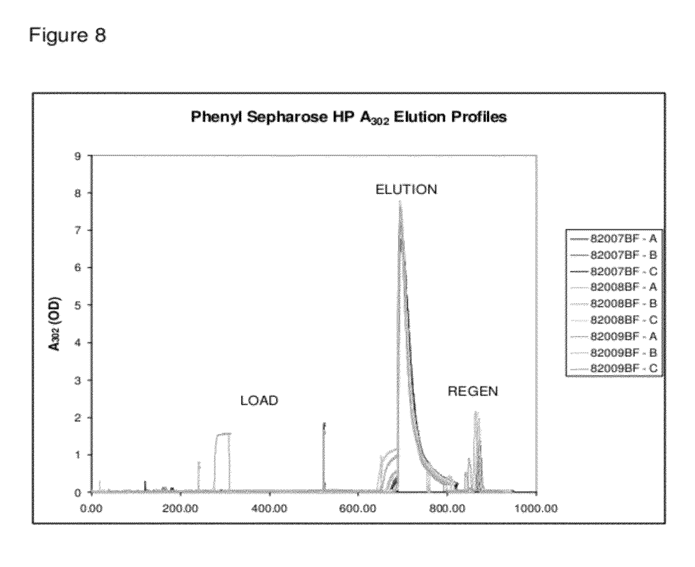

Phenyl Sepharose® HP hydrophobic interaction resin (GE Healthcare) and a Chromaflow® Acrylic chromatography column (GE Healthcare) were utilized for this process step. The phenyl column was 60 cm in diameter with a target height of 15±1 cm (bed volume 42.4 L). The loading limit for the column was 40 grams of sample per liter of Phenyl Sepharose® HP resin. The step was performed at 17±2° C., and at a flow rate of 75 cm/hr. The load material was warmed, when required, to 17±2° C. prior to the start of the first cycle. The column was pre-washed with water and equilibrated with 1.0 M ammonium sulfate and 18 mM sodium phosphate, pH 7.0. Following equilibration, the column was loaded with the diluted phenyl load. After loading, the column was washed to baseline absorbance (A₂₈₀) with 1.1 M ammonium sulfate and 20 mM sodium phosphate, pH 7.0, followed by 0.95 M ammonium sulfate and 17 mM sodium phosphate, pH 7.0, respectively. The product was eluted from the column at a reduced flow rate of 37.5 cm/hr with 0.55 M ammonium sulfate and 10 mM sodium phosphate, pH 7.0, into a portable tank. The eluate was collected from 5 OD on the front to 1 OD on the tail at 280 nm with a 1 cm path length. For each cell culture batch, the column was cycled two additional times to process the approximately 4700 g of protein sample that was expected. Between each cycle, the column was regenerated with water for injection (WFI). The eluate was held at ≦22° C. for 1 day. Optionally, the eluate can be chilled to ≦8° C. and held up to 10 days before proceeding to the nanofiltration step. Phenyl column operations were consistent in regard to elution chromatographic profiles. Overlays are illustrated in FIGS. 7 and 8.

Operational data and yields for Phenyl Sepharose® HP chromatography are detailed in Table 4. Average column loadings were approximately 36 g of protein per L of resin per cycle. Average yield for the Phenyl Sepharose® step was 89%.

TABLE 4
Summary of Phenyl Sepharose ® HP Chromatography

Run #

| | 1 | 2 | 3 | AVG | STD |
|---|---|---|---|---|---|
| Load Amount (g) | 4509.5 | 4777.4 | 4481.1 | 4589.3 | 163.5 |
| Column Loading | 35 | 38 | 35 | 36 | 1 |
| (g sample/L resin) | | | | | |
| Eluate Pool (L) | 396.7 | 363.3 | 332.9 | 364.3 | 31.9 |
| Eluate Pool Conc. (g/L) | 11.12 | 11.45 | 10.52 | 11.03 | 0.47 |
| Step Yield (%) | 98 | 89 | 81 | 89 | 9 |

### Nanofiltration

Nanofiltration was used to remove adventitious viruses 20 nm in diameter that might potentially be present in the Phenyl Sepharose® HP purified material. The nanofiltration filter train was comprised of two Pall 0.15 m² 0.1 μm Fluorodyne® II PVDF capsule filters (total of 0.3 m² nominal filter area) as protecting filters for two 20-inch Sartorius Virosart® CPV filters (total of 2.8 m² nominal filter area) or two 20-inch Pall DV20 filters in parallel. The step was performed at 10-14° C. To monitor the filtration, pressure gauges were mounted upstream of the prefilter and upstream of each nanofilter housing. During the filtration, the pressure was held at ≦32 psig. After all of the Phenyl eluate had been filtered, the filter train was rinsed with 25 kg of 15 mM histidine, pH 6.0, to recover any protein sample which may have been retained in the filter housings. For each cell culture batch, one nanofiltration was performed. The filtrate was held at ≦22° C. up to 1 day or chilled to ≦8° C. and held up to 10 days before proceeding to the formulation step.

Average yield for the nanofiltration operation was 99%. Average filter loading for the Sartorius filters was 130 L/m² per run (equivalent to 1413 g/m² per run). The DV20 loading was 61 L/m² per run (equivalent to 693 g/m² per run). Filtration operations were consistent based on filtrate volumes, filtrate concentrations and yields. Operation and yields are detailed in Table 5.

TABLE 5
Summary of Nanofiltration Operation

| | Run # | | | | |
|---|---|---|---|---|---|
| | 1 | 2 | 3 | AVG | STD |
| Viral Filter Nominal Area | 2.8 | 6.0 | 2.8 | n/a | n/a |
| (m²) | | | | | |
| Viral Filter Loading (g of | 1575 | 693 | 1251 | 1413^{a} | 230^{a} |
| sample/m² of filter area) | | | | | |
| Load Volume (L) | 396.7 | 363.3 | 332.9 | 364.3 | 31.9 |
| Viral Filter Loading (L of | 142 | 61 | 119 | 130^{a} | 16^{a} |
| sample/m² of filter area) | | | | | |
| Rinse Volume (kg) | 25 | 25 | 25 | 25 | 0 |
| Filtrate Volume (L) | 407.2 | 381.6 | 354.3 | 381.0 | 26.5 |
| Filtrate Concentration (g/L) | 10.28 | 10.73 | 10.31 | 10.4 | 0.3 |
| Step Yield (%) | 95 | 98 | 104 | 99 | 5 |

^{a}Values calculated using data relevant to Sartorius filters in Runs 1 and 3 only.

### Formulation (Ultrafiltration and Diafiltration)

Each lot of viral filtrate was concentrated and formulated by ultrafiltration and diafiltration. Three Millipore Pellicon® 2 Biomax UF Modules with a 30 kD molecular weight cut off and 2.5 m² surface area each (total of 7.5 m² nominal filter area) were used for the first portion of the formulation operation. The step was performed at 10-14° C. The viral filtrate was first concentrated to a target of 70 g/L by ultrafiltration. Next, continuous diafiltration with a minimum of 8 volumes of 19 mM histidine, pH 5.6, was performed. After diafiltration, the drug substance was further concentrated to a target of 195 g/L. The ultrafiltration system was then drained of product and rinsed with approximately 8 kg of 19 mM histidine, pH 5.6, to recover product held up in the system. The concentrate and wash were combined to produce a diafiltered sample with a target concentration of 130-150 g/L. The formulated concentrate was then filtered through one Sartopore® 2, 800 sterile capsule filter into a holding tank. The filtrate was held for up to 7 days at ≦22° C. before proceeding to the final bottling step.

Average yield for the formulation operation was 99%. Formulation operations were consistent based on final retentate volumes, concentrations and yields (see Table 6).

TABLE 6
Summary of Formulation Operation

Run #

| | 1 | 2 | 3 | AVG | STD |
|---|---|---|---|---|---|
| Initial Amount (g) | 4186 | 4095 | 3653 | 3978 | 285 |
| Retentate Volume (L) | 30.9 | 26.1 | 24.0 | 27.0 | 3.5 |
| Retentate Concentration | 140.1 | 149.9 | 149.7 | 146.6 | 5.6 |
| (g/L) | | | | | |
| Retentate Amount (g) | 4326 | 3911 | 3587 | 3941 | 370 |
| Step Yield (%) | 103 | 96 | 98 | 99 | 4 |

### Filtration, Bottling, and Freezing

The bottling operations were performed in a flow hood at 2-8° C. The sample was pumped through a Millipak® 200 0.22 μm filter into pre-sterilized, pyrogen-free polyethylene terephthalate glycol containers. Approximately 1.6 L was filled per 2 L bottle. Within three hours of the end of the bottling operation, the filled labeled bottles were frozen at −80° C.

Average yield for the final bottling operation was 99%. The bottling operations were consistent based on protein concentration, protein amounts, and final yields (see Table 7).

TABLE 7
Summary of Sterile Filtration, Bottling, and Freezing Operations

| | Run # | | | | |
|---|---|---|---|---|---|
| | 1 | 2 | 3 | AVG | STD |
| Starting Amount (g) | 4287 | 3932 | 3564 | 3928 | 362 |
| Bulk Product Concentration (g/L) | 138 | 150 | 148 | 145 | 6 |
| Bulk Drug Amount (g) | 4234 | 3866 | 3517 | 3872 | 359 |
| Step Yield (%) | 99 | 98 | 99 | 99 | 1 |

### Yield Summary

The yields from each process step are given in Table 8. The reactor end amount and the bottled bulk drug substance amount were used to calculate the overall yield. The average calculated overall yield was 60%. When corrected for in-process sampling, the average calculated overall yield was 68%.

TABLE 8
Summary of yields for MAb A purification

| Steps | 1 | 2 | 3 | AVG | STD |
|---|---|---|---|---|---|
| Primary Harvest (%) | 91 | 89 | 92 | 91 | 2 |
| ProSep ® Ultra Plus Capture (%) | 91 | 91 | 89 | 90 | 1 |
| Viral Inactivation/Pod/Q Membrane | 91 | 100 | 97 | 96 | 5 |
| (%) | | | | | |
| Phenyl Sepharose ® HP | 98 | 89 | 81 | 89 | 9 |
| Chromatography (%) | | | | | |
| Viral Filtration (%) | 95 | 98 | 104 | 99 | 5 |
| UF/DF (%) | 103 | 96 | 98 | 99 | 4 |
| Bottling (%) | 99 | 98 | 99 | 99 | 1 |
| Overall Process Yield (Corrected for | 72 | 67 | 65 | 68 | 3 |
| Sampling) (%) | | | | | |

### Product Quality

Final bulk drug substance was tested for a full panel of quality attributes. Overall, the three batches of final drug substance were consistent and within specifications for all attributes tested (see Table 9).

TABLE 9
Product purity in final drug substance for MAb A.

| Assays | Run 1 | Run 2 | Run 3 |
|---|---|---|---|
| Monomer % | 99 | 99 | 99 |
| Host Cell Protein (ng/mg) | <0.21 | <0.21 | 0.34 |
| Protein A (ng/mg) | 0.05 | 0.05 | 0.06 |
| DNA (pg/mg) | <1 | <1 | <1 |

### Example 2

In this example, a protein purification process very similar to that described in Example 1 was performed to purify MAb B. Differences between the two processes are described herein. If an aspect of the process is not described in detail, it is as described for Example 1.

### Primary Recovery

Centrifugation and depth filtration served as the primary recovery steps. The centrifugation process was the same as described for Example 1. The centrifuged harvest was then passed through a filter train that consisted of ten 1.1 m² Millipore® X0HC media Pod units. The sample was then filtered through three 30-inch Sartopore® 2 0.45/0.2 μm filters, in series. After the sample was filtered, it was rinsed with 200 kg of 25 mM Tris, 100 mM sodium chloride, pH 7.2 followed by air blown to remove the remaining filtrate.

Centrifugation and filtration of the harvest were performed as a single unit operation. The filtrate was collected in a 3000 L harvest tank, chilled to 4-12° C., and held for up to 5 days.

### Protein A Capture Chromatography

The protein A capture step of Example 2 was substantially similar to that described in Example 1. The loading limit for the column was 43 grams of MAb B per liter of Protein A resin. Eight to nine cycles were completed for each batch. The step was performed at ambient temperature and used a 2-step linear velocity loading of 600 cm/hr up to 30 g/L and 400 cm/hr up to 43 g/L. 0.15 M phosphoric acid (pH 1.5) was used for regeneration of every cycle. 6 M urea was used for cleaning, every five cycles and at the end of the process. 50 mM NaAcetate, pH 5, 2% benzyl alcohol was used for sanitization and storage.

### Viral Inactivation, Depth Filtration, and Q Membrane Chromatography

The next step in the process is the combination step which includes viral inactivation, depth filtration, and chromatography. In this step, the low pH inactivation was accomplished in the manner set forth in Example 1. Following inactivation, the sample was flowed through an 8.8 m² X0HC Pod followed by two 780 mL Mustang® Q membrane adsorber which were set in parallel. The flow rate through the Q membrane adsorber was 10 CV/min. After depth filtration and again after Q membrane processing, the sample was flowed through a Sartopore® 2 30-inch (0.45 μm+0.2 μm) capsule filter.

### Hydrophobic Interaction Chromatography

A Phenyl Sepharose® HP hydrophobic interaction resin (GE Healthcare) and a Chromaflow® Acrylic chromatography column (GE Healthcare) were utilized for this process step. The phenyl column was 80 cm in diameter with a target height of 15±1 cm. Prior to this polishing step, the Q membrane effluent was diluted with 2.2 M ammonium sulfate and 40 mM sodium phosphate, pH 7.0, to obtain a target concentration of 1.1 M ammonium sulfate and 11 mM sodium phosphate and then filtered through a Sartopore® 2 (0.45 μm+0.2 μm) capsule filter prior to loading onto the column. The column was pre-washed with water and equilibrated with 1.1 M ammonium sulfate in 20 mM sodium phosphate, pH 7.0 solution. Following equilibration, the column was loaded with the diluted phenyl load at 75 cm/hr flow rate. After loading, the column was washed to baseline absorbance (A₂₈₀) with 1.4 M ammonium sulfate and 25 mM sodium phosphate, pH 7.0. The product was eluted from the column at a reduced flow rate of 37.5 cm/hr with 0.625 M ammonium sulfate and 11 mM sodium phosphate, pH 7.0. The eluate was collected from 1 OD on the front to 1 OD on the tail at 280 nm with a 1 cm path length. The sample was processed through the column in two cycles. The loading limit for the column was 64 grams of sample per liter of Phenyl Sepharose® HP resin.

### Nanofiltration

The nanofiltration filter train was comprised of a Sartorius 0.1 μm Maxicap® filter as a pre-filter for two 20-inch Sartorius Virosart® CPV filters (total of 2.8 m² nominal filter area) in parallel. During the filtration the pressure was held at 34 psig.

### Formulation (Ultrafiltration and Diafiltration)

Each lot of viral filtrate was concentrated and formulated by ultrafiltration and diafiltration. The Millipore Pellicon® 2 Biomax UF Modules with a 30 kD molecular weight cut off (total membrane area of 10 m²) were used for the first portion of the formulation operation. The viral filtrate was first concentrated to a target of 50 g/L by ultrafiltration. Next, continuous diafiltration with a minimum of 8 volumes of 23 mM histidine, pH 5.6, was performed. After diafiltration, the drug substance was further concentrated to a target of 180 g/L. The ultrafiltration system was then drained of product and rinsed with approximately 6-8 kg of 15 mM histidine, pH 5.6, to recover product held up in the system. The concentrate and wash were combined to produce a diafiltered sample with a target concentration of 120-160 g/L,

Filtration, bottling, and freezing were accomplished as set forth in Example 1.

The purification yields and final product quality for MAb B were summarized in Table 10 and 11. Four batches were run successfully with average total purification yield of 69%. The impurity levels in the final bulk drug substances of all the batches were comparable, and met the product quality specification.

TABLE 10
Summary of Yields for MAb B purification.

| Run # | 1 | 2 | 3 | 4 | AVG | STD |
|---|---|---|---|---|---|---|
| Clarification (%) | 96 | 91 | 89 | 95 | 93 | 3 |
| ProSep ® Ultra Plus | 96 | 95 | 93 | 91 | 94 | 2 |
| Capture (%) | | | | | | |
| Viral Inactivation/Pod/Q | 90 | 87 | 91 | 92 | 90 | 2 |
| Membrane (%) | | | | | | |
| Phenyl Sepharose ® HP | 89 | 93 | 95 | 90 | 92 | 3 |
| Chromatography (%) | | | | | | |
| Viral Filtration (%) | 99 | 102 | 97 | 101 | 100 | 2 |
| UF/DF (%) | 103 | 92 | 98 | 95 | 97 | 5 |
| Bottling (%) | 99 | 100 | 98 | 100 | 99 | 1 |
| Overall Process Yield | 75 | 65 | 67 | 69 | 69 | 4 |
| (Corrected for Sampling) (%) | | | | | | |

TABLE 11
Product purity in final drug substance for MAb B.

| Assays | Run 1 | Run 2 | Run 3 | Run 4 |
|---|---|---|---|---|
| Monomer (%) | 99.7 | 99.8 | 99.6 | 99.4 |
| Host Cell Protein (ng/mg) | <0.14 | <0.14 | <0.14 | 0.14 |
| Protein A (ng/mg) | <0.29 | <0.29 | <0.29 | <0.29 |
| DNA (pg/mg) | <1 | <1 | <1 | <1 |

### Example 3

In this example, another protein purification process was performed to purify MAb A in lab scale. The X0HC filtrate from the third batch run, as described in Example 1, was adjusted to pH 8.1 by adding 1M Tris, pH 9.5 solution and the conductivity was adjusted to 9 mS/cm by adding 1M NaCl. Approximately 270 mL adjusted filtrate was then flowed through three 0.18 ml Acrodisc® Mustang® Q membrane adsorber devices in parallel. The conductivity of the Q membrane flow-through pool was adjusted further to 9 mS/cm by adding 1M NaCl and then 0.22 μm filtered. This conditioned pool was then flowed through a 5 mL prepacked Capto® adhere column at 3 min residence time flow rate. The load level on the Capto® adhere column was 221 mg/ml and a 20 CV of equilibration buffer wash was performed following the feed load. The product pool was collected based on UV280 reading from 200 mAU during product load to 200 mAU during buffer wash. The experiment was conducted at room temperature. The concentration and volume of the Capto® adhere product pool were measured to calculate the step yield, and the pool was analyzed for aggregates/monomer using SEC, and HCP and protein A levels using in-house ELISA assays.

The lab scale Q membrane flow-through showed step yield of 93-97%, and the Capto® adhere column polishing step gave a step yield of 89%. Thus the total process yield using Capto® adhere for final polishing is similar to that using Phenyl Sepharose® HP as shown in Example 1. In addition, the quality of the product pool following Capto® adhere purification also met product specification, as shown in Table 12.

TABLE 12
Purification performance for MAb A through Protein A capture
followed by POD filtration/Q membrane flow-through and
Capto ® adhere flow-through polishing.

| | Impurity levels in Capto ® |
|---|---|
| Assays | adhere FTW pool |
| Monomer % | 99.8 |
| Host Cell Protein (ng/mg) | 3.5 |
| Protein A (ng/mg) | 0.01 |

### Example 4

In this example, a protein purification process similar to that described in Example 3 was performed to purify MAb B in lab scale. The Q membrane flow-through pool from the second batch run, as described in Example 2, was adjusted to pH 8.1 by adding 1M Tris, pH 9.5, and the conductivity was adjusted to 6 mS/cm by adding 1M NaCl before filtering through a 0.22 μm membrane. This conditioned pool was then flowed through a 5 mL prepacked Capto® adhere column at 3 min residence time flow rate. The load level on the Capto® adhere column was 256 mg/ml, and a 20 CV of equilibration buffer wash was performed following the feed load. The product pool was collected based on UV280 reading from 200 mAU during product load to 200 mAU during buffer wash. The experiment was conducted at room temperature. The concentration and volume of the Capto® adhere product pool were measured to calculate the step yield, and the pool was analyzed for aggregates/monomer using SEC, and HCP and protein A levels using in-house ELISA assays.

The Capto® adhere column polishing step gave a step yield of 91.6%, which is similar to the Phenyl Sepharose® HP bind-elute step shown in Example 2. In addition, the quality of the product pool following Capto® adhere purification met product specification, as summarized in Table 13.

TABLE 13
Purification performance for MAb B through Protein A capture
followed by POD filtration/Q membrane flow-through and
Capto adhere flow-through polishing.

| | Impurity levels in Capto |
|---|---|
| Assays | adhere FTW pool |
| Monomer % | 99.0 |
| Host Cell Protein (ng/mg) | 3.4 |
| Protein A (ng/mg) | 0.0 |

### Example 5

In this example, a protein purification process similar to that described in Example 4 was performed to purify MAb B in lab scale. The X0HC filtrate from the second batch run, as described in Example 2, was adjusted to pH 6.5 by adding 1M Tris, pH 9.5, and the conductivity was adjusted to 6 mS/cm by adding 1M NaCl or diluting with Milli-Q® water before filtering through a 0.22 μm membrane. This conditioned pool was then flowed through a 5 mL prepacked PPA HyperCel™ column at 3 min residence time flow rate. Two runs were conducted. The load levels on the PPA HyperCel™ column were 104 and 235 mg/ml respectively, and a 20 CV of equilibration buffer wash was performed following each feed load. The product pool was collected based on UV280 reading from 200 mAU during product load to 200 mAU during buffer wash. The experiment was conducted at room temperature. The concentration and volume of the PPA HyperCel™ product pool were measured to calculate the step yield, and the pool was analyzed for aggregates/monomer using SEC, and HCP and protein A levels using in-house ELISA assays.

The feed for this experiments contains about 98.1% monomer (1.7% aggregates), 7 ng/mg HCP and spiked with 23.6 ng/mg protein A. The performance of the PPA HyperCel™ resin was summarized in Table 14. The yield at higher loading level (235 mg/ml) was 92%, comparable to that of Phenyl Sepharose® HP polishing step shown in Example 2. Also, the quality of the product pools following PPA HyperCel™ purification met product specification. Since the load for this run did not go through the Q membrane, it is expected that the product quality will be further improved if the Q membrane is used between the X0HC filtration and PPA hypercel polishing step.

TABLE 14
Purification performance for MAb B through Protein A capture
followed by POD filtration and PPA Hypercel ™ flow-through polishing.

Impurity levels in PPA
hypercel FTW pool

| Test | 100 mg/ml load | 235 mg/ml load |
|---|---|---|
| Monomer % | 99.2 | 99.0 |
| Host Cell Protein (ng/mg) | 2.31 | 3.62 |
| Protein A (ng/mg) | 0.02 | 0.03 |

### Example 6

In this example, another protein purification process was performed to purify MAb B in lab scale. The protein A eluate as described in Example 2, was adjusted to pH 5 by adding 1M Tris, pH 9.5 solution and the conductivity was adjusted to 8 mS/cm by adding 1M NaCl followed by 0.22 μm filtration. This conditioned material was then flowed through a 8 mL Poros XS® (Life Technologies) cation exchange column at 4 min residence time flow rate. Prior to load, the column was cleaned with 0.1 M NaOH, equilibrated with 50 mM sodium acetate, 35 mM NaCl, pH 5 buffer. After loaded with 72 mg/ml MAb B, the column was washed with equilibration buffer and then eluted with 50 mM sodium acetate, 220 mM NaCl, pH 5 buffer. The eluate was collected based on UV280 reading from 200 mAU to 200 mAU. The experiment was conducted at room temperature. The concentration and volume of the Poros XS® product pool were measured to calculate the step yield, and the pool was analyzed for aggregates/monomer levels using SEC, and HCP and protein A levels using in-house ELISA assays.

Table 15 summarizes the purification performance for this polishing step. A step yield of almost 100% was obtained and all the impurity levels are within product specifications Since the load for this run did not go through the X0HC POD and Q membrane polishing step, it is expected that the product quality will be further improved when these steps are incorporated.

TABLE 15
Poros XS cation exchange column polishing performance for MAb B
protein A eluate.

| | | | Protein A |
|---|---|---|---|
| | Monomer (%) | HCP (ng/mg) | (ng/mg) |
| Feed | 95 | 514 | 8.6 |
| Eluate | 99.5 | 4 | 0.4 |

### Example 7

In this example, another protein purification process was performed to purify MAb C in lab scale. Low pH viral inactivated, Millipore POD depth filtered material, as described in Example 1, was adjusted to pH 5 by adding 2 M acetic acid to the solution and the conductivity was adjusted to 5 mS/cm by diluting with water followed by 0.22 μm filtration. This conditioned material was spiked with additional amounts of Protein A and host cell proteins to examine the capacity of this chromatography resin to remove these process impurities. The spiked material was loaded onto a 4.9 mL Poros XS® (Life Technologies) cation exchange column at 2.9 min residence time flow rate. Prior to load, the column was cleaned with 0.1 M NaOH, equilibrated with 100 mM sodium acetate, pH 5 buffer. After loading with 68 mg/ml MAb C, the column was washed with equilibration buffer and then eluted with 380 mM sodium acetate, pH 5 buffer. The eluate was collected based on UV280 reading from 200 mAU to 400 mAU. The experiment was conducted at room temperature. The concentration and volume of the Poros XS® product pool were measured to calculate the step yield, and the pool was analyzed for aggregates/monomer levels using SEC, and HCP and protein A levels using in-house ELISA assays.

Table 16 summarizes the purification performance. A step yield of 93% was obtained and all the impurity levels are within product specifications.

TABLE 16
Poros XS cation exchange column polishing performance for MAb C
protein A eluate.

| | Aggregates | Monomer | HCP | Protein A |
|---|---|---|---|---|
| | (%) | (%) | (ng/mg) | (ng/mg) |
| Load | 1.1 | 98.9 | 62.3 | 38.7 |
| Eluate | 0.4 | 99.5 | 0.8 | 3.5 |

All references cited in this specification, including without limitation, all papers, publications, patents, patent applications, presentations, texts, reports, manuscripts, brochures, books, internet postings, journal articles, and/or periodicals are hereby incorporated by reference into this specification in their entireties. The discussion of the references herein is intended merely to summarize the assertions made by their authors and no admission is made that any reference constitutes prior art. Applicants reserve the right to challenge the accuracy and pertinence of the cited references.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged in whole or in part. Furthermore, those of ordinary skill in the art will appreciate that the foregoing description is by way of example only, and is not intended to limit the invention so further described in such appended claims. Therefore, the spirit and scope of the appended claims should not be limited to the description of the versions contained therein.

What is claimed is:

1. A method for purifying a protein comprising:
a. providing a sample containing the protein;
b. processing the sample through a capture chromatography resin to provide a first eluate comprising the protein;
c. inactivating viruses in the first eluate to provide an inactivated eluate comprising the protein;
d. processing the inactivated eluate through at least one depth filter to provide a filtered eluate comprising the protein; and
e. processing the filtered eluate through at least one ion-exchange membrane to provide a second eluate comprising the protein.

2. The method of claim 1 wherein the depth filtration step and the ion-exchange membrane step are provided in a filter train.

3. The method of claim 1 wherein the capture chromatography resin is selected from the group consisting of an affinity resin, an ion exchange resin, mixed-mode resin, and a hydrophobic interaction resin.

4. The method of claim 1 wherein the capture chromatography resin is selected from the group consisting of a protein A resin, a protein G resin, a protein A/G resin, and a protein L resin.

5. The method of claim 1 wherein the protein is selected from the group consisting of a protein fragment, an antibody, a monoclonal antibody, an immunoglobulin, and a fusion protein.

6. The method of claim 1 wherein the sample is a cell culture.

7. The method of claim 1 wherein the sample is clarified prior to processing through the capture chromatography resin.

8. The method of claim 7 wherein the sample is clarified by a clarification method selected from the group consisting of centrifugation, microfiltration, ultrafiltration, depth filtration, sterile filtration, and treatment with a detergent.

9. The method of claim 1 wherein the viral inactivation comprises a method selected from the group consisting of treatment with acid, detergent, chemicals, nucleic acid cross-linking agents, ultraviolet light, gamma radiation, and heat.

10. The method of claim 9 wherein viral inactivation comprises lowering the pH of the first eluate to a pH of from about 3 to about 4.

11. The method of claim 10 wherein the first eluate is incubated for about 30 to about 90 minutes during viral inactivation.

12. The method of claim 1 wherein the inactivated eluate is adjusted to pH 5 to 10 before depth filtration step.

13. The method of claim 1 wherein the depth filtration step comprises filtration through at least one depth filter.

14. The method of claim 1 wherein the depth filtration step comprises filtration through at least two depth filters arranged in series or in parallel.

15. The method of claim 1 wherein the depth filtration step is followed by a capsule sterile filtration step.

16. The method of claim 1 wherein the ion-exchange membrane comprises a Q membrane.

17. The method of claim 16 wherein the Q membrane step is conducted in flow-through mode.

18. The method of claim 1 wherein the ion-exchange membrane step is followed by a capsule sterile filtration step.

19. The method of claim 1 wherein the inactivated eluate is processed through one depth filter and the filtered eluate is processed through the ion-exchange membrane in series.

20. The method of claim 1 wherein the second eluate is further subjected to an additional chromatography step.

21. The method of claim 20 wherein the additional chromatography step is selected from the group consisting of hydrophobic interaction chromatography, mixed mode chromatography, and cation exchange chromatography.

22. The method of claim 1 wherein the second eluate is further subjected to a nanofiltration step.

23. The method of claim 1 wherein the second eluate is further subjected to an ultrafiltration and diafiltration step.

24. A method for purifying a protein comprising:
a. providing a sample containing the protein;
b. clarifying the sample to provide a clarified sample;
c. processing the clarified sample through a capture chromatography resin to provide a first eluate comprising the protein;
d. inactivating viruses in the first eluate to provide an inactivated eluate comprising the protein;
e. processing the inactivated eluate through at least one depth filter to provide a filtered eluate comprising the protein;
f. processing the filtered eluate through at least one ion-exchange membrane to provide a second eluate comprising the protein;
g. processing the second eluate through an additional chromatography resin to provide a third eluate comprising the protein;
h. subjecting the third eluate to nanofiltration to provide a nanofiltered eluate comprising the protein; and
i. subjecting the nanofiltered eluate to ultrafiltration and diafiltration.

25. The method of claim 24 wherein the additional chromatography resin comprises a mixed-mode chromatography resin.

26. The method of claim 25, wherein the processing of the second eluate through the additional mixed-mode chromatography resin comprises one or more chromatography techniques selected from the group consisting of anion exchange, cation exchange, hydrophobic interaction, hydrophilic interaction, hydrogen bonding, pi-pi bonding, and metal affinity.

27. The method of claim 26 wherein the processing of the second eluate through the additional mixed-mode chromatography resin comprises a combination of anion exchange and hydrophobic interaction chromatography mechanisms.

28. The method of claim 26 wherein the mixed-mode chromatography column can be operated in flow-through or bind-elute mode.

29. The method of claim 24 wherein the additional chromatography resin comprises a cation exchange resin.

30. The method of claim 29, wherein the processing of the second eluate through the additional mixed-mode chromatography resin comprises one or more chromatography techniques selected from the group consisting of anion exchange, cation exchange, hydrophobic interaction, hydrophilic interaction, hydrogen bonding, pi-pi bonding, and metal affinity.

31. The method of claim 30 wherein the processing of the second eluate through the additional mixed-mode chromatography resin comprises a combination of anion exchange and hydrophobic interaction chromatography mechanisms.

32. The method of claim 29 wherein the cation exchange chromatography column is operated in bind-elute mode.
